(19) United States
(12) Patent Application Publication
(10) Pub. No.: US 2025/0312787 A1
(43) Pub. Date: Oct. 9, 2025

(54) FLUIDIC CARTRIDGE KIT WITH DISPENSING CAP

(71) Applicant: AXXIN PTY LTD, Fairfield, Victoria (AU)

(72) Inventors: Gavin RAMANATHAN, Fairfield (AU); Anthony DO, Fairfield (AU); Jack Maxwell HOPPER, Fairfield (AU); William Robb HOPPER, Fairfield (AU); Jonathan Scott RICHARDS, Fairfield (AU); Tom DEBENHAM, Fairfield (AU)

(21) Appl. No.: 18/862,923

(22) PCT Filed: May 3, 2023
(86) PCT No.: PCT/AU2023/050370

§ 371 Date: Nov. 4, 2024

(30) Foreign Application Priority Data
May 3, 2022 (AU) 2022901155

(51) Int. Cl. B01L 3/00; A61B 5/15

(52) U.S. Cl. CPC B01L 3/502715; A61B 5/150343; A61B 5/150351; A61B 5/150755; B01L 2200/16; B01L 2200/185; B01L 2300/044; B01L 2300/047; B01L 2300/0654; B01L 2300/0672; B01L 2400/0487

(57) ABSTRACT

A fluidic cartridge kit, including: a fluidic cartridge having an analysis portion and a sample reservoir sealed with a removable shipping closure to contain contents of the sample reservoir during transport: and a dispensing cap; wherein the dispensing cap includes: a closure and dispensing component configured to engage the dispensing cap to the fluidic cartridge and close an opening of the sample reservoir of the fluidic cartridge following removal of the shipping closure from the sample reservoir; a compressible sealing component configured to form a seal between the closure and dispensing component and the sample reservoir and thereby seal the sample reservoir; and a sample release component coupled to the closure and dispensing component and configured to make an opening in a wall of the sealed sample reservoir; and wherein the closure and dispensing component is operable to engage the fluidic cartridge, close the sample reservoir, and compress the compressible sealing component therebetween to seal the sample reservoir, wherein the sample reservoir is partially filled with a sample fluid; wherein the closure and dispensing component is further operable to compress the compressible scaling component and thereby pressurise the sealed sample reservoir and to cause the sample release component to make the opening in the wall of the pressurised sample reservoir; and wherein the closure and dispensing component, the compressible scaling component and the sample release component are configured such that the pressurised sample reservoir is pressurised to a predetermined pressure range that causes at least a portion of the sample fluid to be dispensed through the opening in the pressurised sample reservoir into the analysis portion of the fluidic cartridge.

### TECHNICAL FIELD

The present invention relates to diagnostic and biomedical tests involving biological or environmental samples. More specifically, the present invention relates to a fluidic cartridge kit including a fluidic cartridge and a cap for dispensing a liquid biological or environmental sample into the fluidic cartridge, and a diagnostic test method involving the dispensing cap and fluidic cartridge.

### BACKGROUND

Liquid biological samples (e.g., urine, saliva, blood and other bodily fluids) and environmental samples (e.g., water collected from lakes, reservoirs, aquifers or streams) are often used in biological tests for detecting the presence or absence of one or more protein or nucleic acid target(s).

Known investigative procedures are often performed in fluidic test cartridges, cassettes, chips or slides (herein collectively referred to as “fluidic cartridges”), where the test is performed in a reaction chamber of the fluidic cartridge and the test result is observed through a viewing area or viewing window. Other typical components of fluidic cartridges include a sample reservoir connected directly or indirectly to the reaction chamber, and a vent to allow air to escape from the reaction chamber.

Fluidic cartridges can be relatively simple, in which the cartridge has a single reaction chamber into which a single sample is introduced or can be more complex and can include multiple sample reservoirs, mixing wells, fluidic channels, reaction chambers, pumps or valves, etc. for performing more complicated or multiplexed tests. Further, a fluidic cartridge may have only a single layer, whereby movement of liquid within the cartridge occurs in a single plane, or multiple layers where liquid can move between layers.

Reaction chambers are typically preloaded with soluble, dried or lyophilised reagents, which react with the liquid sample. The assay can utilise colorimetry and/or fluorescence, luminescence or other detection methods. For example, in a visual colorimetric test, the colour of the liquid within the reaction chamber may change, depending on the presence or absence of some target material in a sample, and may be visually observed and interpreted by the person conducting the test. Fluorescence-based tests, however, require an excitation signal to stimulate the emission from within the sample found in the reaction chamber. The resulting emission signal is indicative of the test result. The emission signal is may be detected using a sensor that is filtered to exclude the wavelengths of the excitation signal.

A diagnostic test reading instrument is often used to improve the reliability, repeatability and sensitivity of a test, even where a test has a visually readable result. A cartridge is typically inserted into a reader, which provides the required excitation signals, sensors and interpretive software to read and analyse test results. The instrument may also perform additional functions; for example, heating the test liquid within the cartridge to the ideal temperature(s) for conducting the test, or instrument actuators may control movement of liquid within the cartridge.

Prior to conducting a test, some form of sample preparation is generally required. The sample preparation may include heating the sample to a specified temperature or mixing the sample with sample preparation liquids to prepare the target test material. For example, saliva or nasal samples are often collected using a swab, and then washed with elution buffer solution to remove the target test material from the swab. Lysis buffer or heat treatment can be used to release the target test material from within the cells. In most instances, the target test material is a nucleic acid (e.g., DNA or RNA), which can be detected either cell free in the sample, or associated with a cell (which thereby requires release via heat or lysis treatment).

Sample preparation may be conducted separately to the cartridge or within the cartridge. Where sample preparation is conducted separately to the cartridge, the prepared liquid is introduced into a sample reservoir of the cartridge. Alternatively, the sample preparation may take place within a sample reservoir or sample preparation cavity of the cartridge itself. Sample preparation within a cartridge is particularly desirable in point of care settings, where it is preferable to minimize the amount of test equipment required to prepare the sample and perform a test. For example, the test may be isothermal nucleic acid amplification or PCR.

A number of challenges arise when dispensing the prepared sample liquid into a cartridge. The quantity of liquid dispensed into the cartridge is typically small, and the liquid may be viscous. The surface tension of the liquid may slow or prevent movement of the sample from the sample reservoir into the cartridge. Further, the liquid may be required to move through especially narrow fluidic channels of the fluidic cartridge. Therefore, some force is required when dispensing the liquid. However, if the force applied is too large, rapid expulsion of the liquid into the cartridge is likely to produce air bubbles. If large air bubbles are introduced into the cartridge, more specifically, the reaction chamber, the quantity of the sample available for performing the test will be reduced. This can reduce the sensitivity of the test. Consequently, it is critical that an appropriate, controlled force is applied to dispense a sample liquid into a cartridge.

For reproducibility between tests, the quantity of liquid introduced into the fluidic cartridge and ultimately into the reaction chamber should be consistent. Further, introducing too much liquid, especially with force, may overwhelm features of the fluidic cartridge and result in variability in testing. For example, where a vent is present for removing any air from the reaction chamber, forcing too much liquid into the reaction chamber may cause the ventilation membrane to detach, and the cartridge to leak. A leaking cartridge may expose a person conducting a test to potentially harmful reagents or lead to cross-contamination of the sample.

It is desired to overcome or alleviate one or more difficulties of the prior art, or to at least provide a useful alternative.

Reference to any prior art in the present specification is not an acknowledgement or suggestion that this prior art forms part of the common general knowledge in any jurisdiction, or that this prior art could reasonably be expected to be understood, regarded as relevant and/or combined with any other pieces of prior art by a person skilled in the art.

### SUMMARY

In accordance with some embodiments of the present invention, there is provided a fluidic cartridge kit, including:
a fluidic cartridge having an analysis portion and a sample reservoir sealed with a removable shipping closure to contain contents of the sample reservoir during transport; and
a dispensing cap;
wherein the dispensing cap includes:
   a closure and dispensing component configured to engage the dispensing cap to the fluidic cartridge and close an opening of the sample reservoir of the fluidic cartridge following removal of the shipping closure from the sample reservoir;
   a compressible sealing component configured to form a seal between the closure and dispensing component and the sample reservoir and thereby seal the sample reservoir; and
   a sample release component coupled to the closure and dispensing component and configured to make an opening in a wall of the sealed sample reservoir; and
wherein the closure and dispensing component is operable to engage the fluidic cartridge, close the sample reservoir, and compress the compressible sealing component therebetween to seal the sample reservoir, wherein the sample reservoir is partially filled with a sample fluid;
wherein the closure and dispensing component is further operable to compress the compressible sealing component and thereby pressurise the sealed sample reservoir and to cause the sample release component to make the opening in the wall of the pressurised sample reservoir; and
wherein the closure and dispensing component, the compressible sealing component and the sample release component are configured such that the pressurised sample reservoir is pressurised to a predetermined pressure range that causes at least a portion of the sample fluid to be dispensed through the opening in the pressurised sample reservoir into the analysis portion of the fluidic cartridge.

In some embodiments, the fluidic cartridge kit further includes a locking feature configured to lock the closure and dispensing component to prevent inadvertent release of sample fluid from the fluidic cartridge following dispensing.

In some embodiments, the closure and dispensing component includes a screw thread configured to engage with a corresponding screw thread on an outer surface of the sample reservoir.

In some embodiments, the compressible sealing component is a compressible gasket.

In some embodiments, the compressible gasket comprises silicon foam.

In some embodiments, the compressible gasket is composed of silicon foam.

In some embodiments, a portion of the sample release component is configured to puncture and pass through the wall of the sample reservoir to make the opening therein.

In some embodiments, a cross-sectional shape of the sample release component provides an opening to facilitate fluid flow through the opening in the wall of the sample reservoir with the sample release component through the opening.

In some embodiments, the cross-sectional shape of the sample release component is a cross-shape.

In some embodiments, the closure and dispensing component is variably operable to dispense a variable amount of the sample fluid into the analysis portion of the fluidic cartridge.

In some embodiments, the closure and dispensing component is configured to dispense one or more predetermined amounts of the sample fluid into the analysis portion of the fluidic cartridge.

In some embodiments, the closure and dispensing component has a morphology configured to provide a predetermined enclosed volume within the sample reservoir upon sealing, and to effect a predetermined reduction of that volume upon the further operation to pressurise the sample reservoir to a pressure that causes the predetermined amount of the sample fluid to be dispensed into the analysis portion of the fluidic cartridge.

In some embodiments, the closure and dispensing component is domed to provide increased enclosed volume upon sealing of the sample reservoir.

In some embodiments, the dispensing cap and sample reservoir are configured to provide a predetermined enclosed volume within the sample reservoir upon sealing, and to effect a predetermined reduction of that volume upon operation to pressurise the sample reservoir and cause the predetermined amount of the sample fluid to be dispensed into the analysis portion of the fluidic cartridge.

In some embodiments, the sample reservoir is formed as a body of a plastic material with a second opening covered by a foil or seal composed of a different material, and wherein the sample release component makes the opening in the wall of the pressurised sample reservoir by puncturing the foil or seal.

In some embodiments, the analysis portion of the fluidic cartridge comprises a reaction chamber configured to fill with the sample fluid upon the opening in the wall of the pressurised sample reservoir.

In some embodiments, the analysis portion of the fluidic cartridge comprises multiple reaction chambers configured to fill simultaneously with the sample fluid upon the opening in the wall of the pressurised sample reservoir.

In some embodiments, the analysis portion of the fluidic cartridge comprises multiple reaction chambers configured to fill sequentially with the sample fluid upon the opening in the wall of the pressurised sample reservoir.

In some embodiments, the analysis portion of the fluidic cartridge comprises a ventilation port to allow air to escape from the reaction chamber(s).

In some embodiments, the ventilation port comprises a ventilation membrane or filter that is permeable to air but prevents fluid escape.

In some embodiments, the analysis portion comprises a non-transparent material covering the reaction chambers.

In some embodiments, the analysis portion comprises a transparent material covering the reaction chambers.

In some embodiments, the sample reservoir is integrally formed as a body of a plastic material, and the sample release component makes the opening in the wall of the pressurised sample reservoir by puncturing the plastic wall.

In some embodiments, the shipping closure is a cap is made of a bonded plastic laminate.

In some embodiments, the shipping closure is the dispensing cap.

In some embodiments, the shipping closure is a foil sheet that seals the sample reservoir during transport.

In some embodiments, the contents of the sample reservoir includes a lysis buffer.

In accordance with some embodiments of the present invention, there is provided a diagnostic test method, the method comprising:
introducing or forming a sample fluid containing a biological or environmental sample into or in the sample reservoir of any one of the preceding fluidic cartridges;
applying the dispensing cap of any one of the preceding fluidic cartridges;
operating the closure and dispensing component to engage the closure and dispensing component to the fluidic cartridge, close the sample reservoir, and compress the compressible sealing component therebetween to seal the open volume and sample fluid within the sample reservoir; and
further operating the closure and dispensing component to further compress the compressible sealing component to reduce the enclosed volume within the sample reservoir and thereby pressurise the sealed sample reservoir and to cause the sample release component to make the opening in the wall of the pressurised sample reservoir, wherein the pressurised sample reservoir is pressurised to a predetermined pressure range that causes at least a portion of the sample fluid to be dispensed through the opening in the pressurised sample reservoir into the analysis portion of the fluidic cartridge.

In some embodiments, a single action by a user causes the portion of the sample fluid to be dispensed through the opening in the pressured sample reservoir into the analysis portion of the fluidic cartridge.

Also described herein is a dispensing cap for a fluidic cartridge, the dispensing cap including:
a closure and dispensing component configured to engage the dispensing cap to a fluidic cartridge and close an opening of a sample reservoir of the fluidic cartridge;
a compressible sealing component configured to form a seal between the closure and dispensing component and the sample reservoir and thereby seal the sample reservoir; and
a sample release component coupled to the closure and dispensing component and configured to make an opening in a wall of the sealed sample reservoir; and
wherein the closure and dispensing component is operable to engage the fluidic cartridge, close the sample reservoir, and compress the compressible sealing component therebetween to seal the sample reservoir, wherein the sample reservoir is partially filled with a sample fluid;
wherein the closure and dispensing component is further operable to compress the compressible sealing component and thereby pressurise the sealed sample reservoir and to cause the sample release component to make the opening in the wall of the pressurised sample reservoir; and
wherein the closure and dispensing component, the compressible sealing component and the sample release component are configured such that the pressurised sample reservoir has a pressure that causes at least a portion of the sample fluid to be dispensed through the opening in the pressurised sample reservoir into an analysis portion of the fluidic cartridge.

Also described herein is a fluidic cartridge comprising:
the dispensing cap; a sample reservoir; and
an analysis portion.

### BRIEF DESCRIPTION OF THE DRAWINGS

Some embodiments of the present invention are hereinafter described, by way of example only, with reference to the accompanying drawings, wherein:

FIG. 1 includes isometric views of a dispensing cap and fluidic cartridge of a fluidic cartridge kit in accordance with an embodiment of the present invention, shown (A) with the dispensing cap coupled to the fluidic cartridge and closed, and (B) expanded to show a compressible sealing component of the dispensing cap.

FIG. 2 includes a side view (A) and an isometric view (B) of the dispensing cap of FIG. 1.

FIG. 3 includes cross-sectional side views of the dispensing cap and a portion of the fluidic cartridge, where the dispensing cap is (A) engaged and is sealing but not completely closed, and (B) where the dispensing cap is fully closed.

FIG. 4 is an isometric view of a single channel fluidic cartridge of a fluidic cartridge kit in accordance with an embodiment of the present invention.

FIG. 5 is a flow diagram of a method for dispensing liquid from a sample reservoir into the body of the fluidic cartridge using a dispensing cap according to an embodiment of the present invention.

FIG. 6 is a series of images illustrating a sample preparation workflow involving a fluidic cartridge and a dispensing cap, and performed with a diagnostic test reading instrument, according to an embodiment of the present invention.

FIG. 7 includes isometric view of a fluidic cartridge with either a shipping cap (A) or a dispensing cap (B) in accordance with an embodiment of the present invention.

FIG. 8 includes isometric views of a fluidic cartridge with a removable shipping seal, (A) sealing the opening of the sample reservoir or (B) following removal from the sample reservoir.

FIG. 9 includes isometric view of a fluidic cartridge with either a shipping cap (A) or a dispensing cap (B) in accordance with an embodiment of the present invention.

FIG. 10 is a series of images illustrating a sample preparation workflow for G6PD test using a dispensing cap and fluidic cartridge (A-F) in accordance with an embodiment of the present invention.

FIG. 11 is a photographic image of a single channel fluidic cartridge and dispensing cap during pressure testing, showing how a dispensing cap can be provided with a pressure sensing port to enable pressure monitoring during operation so that the fluidic cartridge and dispensing cap can be configured to ensure precise pressure regulation for efficient and reliable fluid dispensing.

### DETAILED DESCRIPTION

Embodiments of the present invention include a fluidic cartridge kit including dispensing cap and a fluidic cartridge, the dispensing cap including a closure and dispensing component, a compressible sealing component, and a sample release component. The dispensing cap is configured to facilitate the dispensing of a sample fluid from a sample reservoir into an analysis portion of the fluidic cartridge, for use with a diagnostic test instrument to perform a diagnostic test on a biological or environmental sample. As described herein, the dispensing cap allows simultaneous closure of the sample reservoir and controlled dispensing of a volume of fluid from the sample reservoir into the analysis portion of the fluidic cartridge by a user. In particular, the dispensing cap is operable to pressurise the sample reservoir to a predetermined pressure range that causes at least a portion of the sample fluid to be dispensed through an opening in the pressurised sample reservoir into the analysis portion of the fluidic cartridge.

### Dispensing Cap

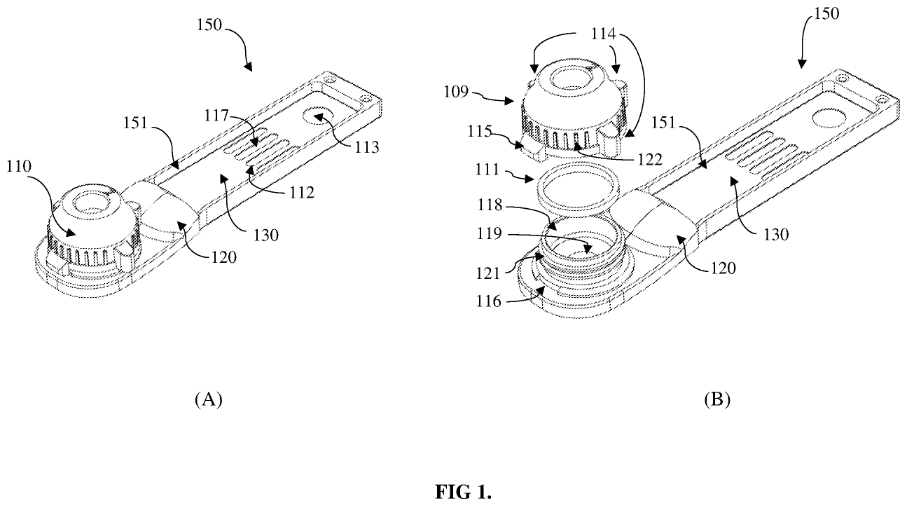

FIG. 1A shows a dispensing cap 110 according to an embodiment of the present invention, coupled to a fluidic cartridge 150 and in a closed and locked position on the fluidic cartridge 150. FIG. 1B is an exploded view of the dispensing cap 110 removed from the fluidic cartridge to show the closure and dispensing component 109, the compressible sealing component 111 being configured to form a seal between the closure and dispensing component 109 and the sample reservoir 118 of the fluidic cartridge 150.

The analysis portion of the fluidic cartridge 151 includes a light shroud 120, and a body 130 which can be manufactured separately to, or integrally formed with, the light shroud 120. In various embodiments, the dispensing cap 110, light shroud 120 and cartridge body 130 may be injection moulded or otherwise formed from a hard plastic or any other suitable material. The light shroud can be made of any suitable non-transparent material known to the skilled person in the art, and is configured to prevent light from interfering with the analysis portion of the fluidic cartridge 151. In the illustrated embodiment, the body 130 is opaque, and includes four (solid) viewing windows 112 for viewing respective reaction chambers 117 located beneath the viewing windows 112. In other embodiments, one, two, three, four or five viewing windows may be provided for viewing, along with one, two, three, four or five reaction chambers located beneath the viewing windows. The reaction chambers 117 are in fluid communication with the sample reservoir 118 via respective fluidic channels (not shown), and with a ventilation port 113 that allows air to escape from the reaction chambers 117. In some embodiments, the reaction chambers 117 are configured to fill simultaneously as the fluid moves from the sample reservoir 118 into the analysis portion 151. Alternatively, in other embodiments, the reaction chambers 117 are configured to fill sequentially as fluid moves from the sample reservoir 118 into the analysis portion 151. The configuration of the reaction chambers 117 simultaneously or sequentially will depend on the test to be performed. The ventilation port 113 may be of any suitable form to allow air to escape from the reaction chambers 117 but should be configured to prevent the escape of fluid from the reaction chamber (or chambers) 117. For example, in some embodiments, the ventilation port comprises a ventilation membrane or filter 508 that is permeable to air but prevents fluid escape. The ventilation port 113 is configured to allow continuous movement of fluid through the reaction chamber 117 (or chambers), and to prevent excess pressure in the reaction chamber (or chambers) 117. The ventilation membrane or filter 508 may be, for example, a Porex PMV301 PTFE porous vent membrane. This is a hydrophobic membrane which maintains pressure within the cartridge up to a pressure of about 20 kPa, above which it will leak (or completely fail if the pressure exceeds about 70 kPa).

Referring to FIG. 1B, the sample reservoir 118 and the analysis portion 151 are initially separated by a barrier (such as a wall or seal) 119 that prevents liquid from flowing from the sample reservoir 118 into the analysis portion 150. The barrier 119 may be a friable seal, for example a foil seal, a thin layer of plastic, or it may be any other suitable frangible or deformable seal known to the skilled person. In some embodiments, the wall may be an integral component or portion of the sample reservoir 118.

Referring to FIG. 1B, the compressible sealing component 111 of the dispensing cap forms a seal between the closure and dispensing component 109 and the reservoir 118. In the described embodiments, the compressible sealing component 111 is a compressible gasket composed of compressible silicone foam or silicone rubber. However, it will be apparent to those skilled in the art that in some other embodiments the compressible gasket can be composed of any other suitable compressible material that does not absorb liquid. In other embodiments, the compressible sealing component 111 may be made of a deformable but substantially incompressible material that changes shape in response to applied pressure.

In embodiments with a compressible gasket composed of a silicone rubber, the gasket is manufactured using injection moulding of liquid silicone rubber (“LSR”). As known by those skilled in the art, LSR is a high purity platinum cured silicone with low compression set, high stability, and an ability to resist extreme heat and cold. LSR comes from a family of thermoset elastomers that have a backbone of alternating silicon and oxygen atoms, and methyl or vinyl side groups. Examples of suitable LSRs include SILASTIC™ Liquid Silicone Rubber (LSR) and SILASTIC™ Fluoro Liquid Silicone Rubber (F-LSR) (Dow). The gaskets are formed using a liquid injection moulding process using a moulding machine that has a metered pumping device, injection unit, and a dynamic or static mixer.

Liquid silicone rubbers are supplied as two separate components (as a kit with “A” and “B” compounds) that are pumped through a static mixer by a metering pump at a 1:1 ratio. One of the components contains the catalyst (“B” compound, typically platinum), and a colouring paste along with other additives may be added before the material enters the static mixer section and forms a homogenous material. From the metering section of the injection moulding machine, the compound is pushed through cooled sprue and runner systems into a heated cavity where the vulcanization takes place. The cold runner and general cooling results in no loss of material in the feed lines. The cooling allows production of LSR parts with nearly zero material waste, eliminating trimming operations and yielding significant savings in material cost. The liquid silicone rubber should have a 20 to 40 Shore-A durometer to be suitable for this application. Further, the gasket should have a rectangular profile and capable of expanding sideways when compressed.

In embodiments with a compressible gasket composed of compressible silicone foam, the gasket can be formed by die cutting or laser cutting a silicone foam sheet (such as those available from Reglin Rubber Australia). The silicone foam gaskets can cut from sheets with adhesive backing on one side to retain the gasket within the cap, where it is protected and does not compress while the cartridge is shipped and stored prior to use. Alternatively, the seal can be retained by friction within the internal rim of the dispensing cap. Silicone foam sheet is moulded with a flat “skin” that forms the upper and lower surfaces of the sheet. The skin forms a good seal on contact with the cap and the sample chamber surface, and the foam core allows significant compression of the gasket to occur. This compression after contact sealing allows the internal sample chamber air volume to be pressurised.

Referring to FIG. 1A and FIG. 1B, the dispensing cap 110 includes one or more (three in the illustrated embodiment) grip tabs 114 positioned about and extending from the generally circular periphery of the closure and dispensing component 109 to provide respective hold points for a user's fingers. Ridges 122 distributed about the periphery are provided to further improve the user's grip.

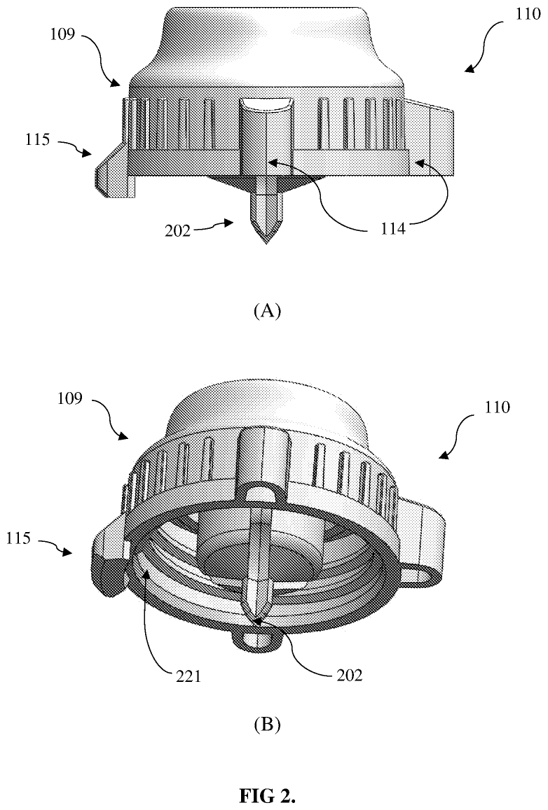

In some embodiments, the dispensing cap 110 includes a locking feature 115 configured to lock the dispensing cap 110 during further operation to prevent inadvertent release of sample fluid from the fluidic cartridge following dispensing. In some embodiments, and as shown in FIGS. 1 and 2, the locking feature 115 is an anti-rotation locking tab shaped to fit into a corresponding locking recess 116 of the sample reservoir 118 of the fluidic cartridge, so that once the dispensing cap 110 is fully closed, it cannot be unscrewed. A locking feature is particularly desirable to ensure that the dispensing cap 110 cannot be inadvertently opened after use; for example, during disposal or transit to a storage facility. Accidental opening of the dispensing cap 110 may potentially expose a person handling the cartridge to hazardous biological or chemical liquids. Alternative locking arrangements will be apparent to those skilled in the art in light of this disclosure.

As shown in FIG. 2B and FIG. 1B, the dispensing cap 110 has a closure and dispensing component 109 which includes an internal thread 221 that corresponds to the thread 121 around the cylindrical outer surface of the sample reservoir 118. In use, either a sample is prepared in the sample reservoir 118, or a pre-prepared sample is transferred into the sample reservoir 118. The closure and dispensing component 109 of the dispensing cap 110 is then screwed onto the sample reservoir 118 compressing the compressible sealing component 111.

As discussed further below, as the dispensing cap 110 via its closing and dispensing component 109 is progressively screwed onto the sample reservoir 118, a position is reached at which the compressible sealing component 111 is in continuous contact with and compressed between the closure and dispensing component 109 and the sample reservoir 118 so that an airtight seal is formed between the closure and dispensing component 109 and the sample reservoir 118. Further turning of the dispensing cap 110 further compresses the compressible sealing component 111, as well as air within the sealed internal space formed by the sample reservoir 118 and the closure and dispensing component 109. The compression of air within the enclosed volume applies pressure to the liquid within the sample reservoir 118. However, the liquid sample cannot be dispensed into the analysis portion 151 until the wall or seal 119 between the sample reservoir 118 and the analysis portion is overcome.

FIGS. 2A and 2B are side and isometric views of the dispensing cap 110 of FIG. 1. A sample release component 202 is coupled to the closure and dispensing component 109 of the dispensing cap 110 and is configured to make an opening in a wall or seal (e.g., foil) of the sealed sample reservoir 119. In the illustrated embodiment, the sample release component 202 is configured to puncture and pass through the wall of the sample reservoir 119 to make an opening herein. The sample release component 202 is configured to further facilitate fluid flow through the opening of the seal or wall 119 of the sample reservoir 118 by rotating the sample release component 202 to create an approximately circular opening. In some embodiments, the sample release component 202 is cross-shaped in cross-section. The sample release component 202 is also configured so that it is long enough to create an opening in the wall or seal 119, but not so long as to pierce through or otherwise cause damage to any other part of the fluidic cartridge. In an embodiment, the sample release component 202 is v-shaped in cross-section and have a piercing tip, but other suitable cross-sectional shapes for the sample release component 202 and alternative configurations for overcoming the wall or seal 119 will be apparent to the skilled person in light of this disclosure. Referring to FIG. 2B, internal thread 221 is used to screw the dispensing cap 110 onto the reservoir 118 thereby closing the reservoir 118.

### Fluidic Cartridge

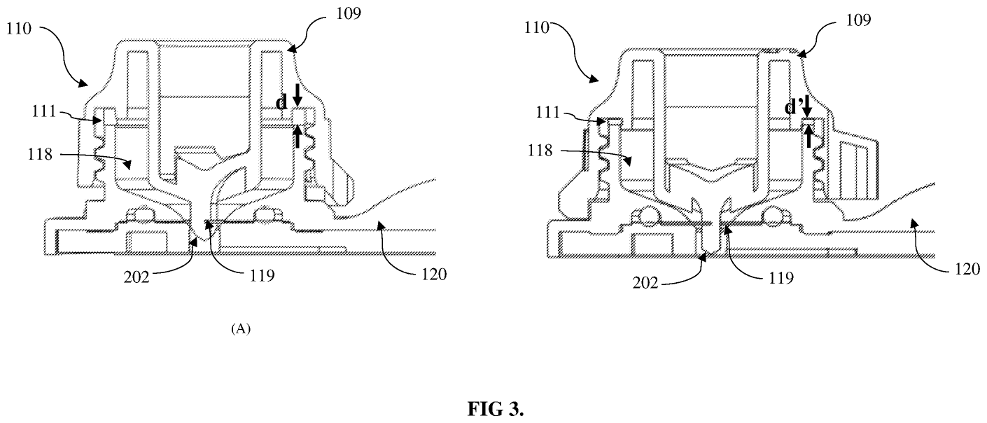

FIGS. 3A and 3B are cross-sectional side views of the dispensing cap 110 and part of the analysis portion 151 of a fluidic cartridge at different stages of operation of the dispensing cap 110. In FIG. 3A, the dispensing cap 110 is partially screwed on, and the compressible sealing component 111 of the dispensing cap 110 is creating a seal between the upper portion of the sample reservoir 118 and the inside of the closure and dispensing component 109. The sample release component 202 has penetrated and created an opening in the wall or seal 119 of the sample reservoir 118. In FIG. 3B, the dispensing cap 110 is fully closed, and the compressible sealing component 111 has been compressed. The compression of the compressible sealing component 111 is indicated by comparison of the arrows labelled d and d′ in FIG. 3A and FIG. 3B, respectively. The change between FIG. 3A d and FIG. 3B d′ represents a reduction in the enclosed volume within the sealed sample reservoir 118 and the dispensing cap 110. The reduction in the enclosed volume increases the pressure in this cavity, which provides the necessary force to move at least a portion of the fluidic sample from the sample reservoir 118 to the analysis portion 151 once the opening in the wall or seal 119 of the sample reservoir 118 is made by the sample release component 202. In the described embodiments, a pressure of at least about 3 kPa is required to expel air from the analysis portion 151 through the ventilation port 113 and cause the fluidic sample to flow from the sample reservoir 118 into the analysis portion 151. It will be appreciated that as the fluidic sample flows from the sample reservoir 118, the pressure in the latter is correspondingly reduced. Accordingly, the overall volume of the sample reservoir 118 that flows into the analysis portion 151 is dependent upon the pressure range in the sample reservoir 118 during that flow, which in turn is determined by the configuration of the dispensing cap (for a given sample reservoir and fluidic cartridge).

The height of the compressible sealing component 111 in its uncompressed state can affect how early in the closing/dispensing process an airtight seal is created between the closure and dispensing component body 109 and the sample reservoir 118, which in turn affects how much pressure is applied to the sample fluid within the sample reservoir 118 as the dispensing cap 110 is screwed onto the fluidic cartridge. As described above, it is essential that the pressure applied to the fluid is sufficiently high (about 4 to 8 kPa in the described embodiments) to force the fluid through or into the analysis portion 151 of the fluidic cartridge 150, but not so high as to create bubbles in the sample fluid or to exceed the operating limits of the cartridge components (about 20 kPa in the described embodiments). Limitations in the manufacturing processes of suitable compressible sealing components in the form of compressible gaskets (for example, the minimum feasible height of a gasket or the height increments available) may limit how precisely this pressure can be controlled.

Through experimentation, it has been determined that varying the amount of air within the sealed sample reservoir 118 can effectively be used to ‘tune’ the compressible sealing component 111. That is, increasing the enclosed volume within the scaled sample reservoir 118 reduces the relative effect of the compression of the compressible sealing component 111 on pressurising the sample reservoir 118. Similarly, reducing the enclosed volume within the sample reservoir 118 increases the relative effect of the compressible scaling component 111. In some embodiments, the compressible scaling component is a compressible gasket which allows a change in height of 1 mm, 2 mm, 3 mm, 4 mm or 5 mm. In one embodiment, the compressible gasket 111 allows a change in height of 2.5 mm. The compression of air within the sample reservoir 118 also dampens pressure spikes that occur as the dispensing cap 110 is screwed down, which otherwise could damage the cartridge (for example, by rupturing the ventilation port 113).

In some embodiments of the dispensing cap 110, the volume within the scaled sample reservoir 118 is increased by doming the top of the dispensing cap 110 and/or by increasing the width of the dispensing cap 110. However, it is less desirable to increase the width of the dispensing cap 110 because the width of the sample reservoir 118 and the light shroud 120 would need to increase accordingly, thereby increasing the amount of material required to produce each cartridge, and consequently the cost of each cartridge. In alternative embodiments, a smaller internal volume is created by providing the closure and dispensing component 109 with, for example, one or more internal concave portions and/or by decreasing its width. For the purposes of manufacturing, the skilled person would understand that the internal volume can be adjusted by altering the size and shape of the closure and dispensing component 109. Various exemplary embodiments of the present invention with differing compressible scaling components 111, larger and smaller sample reservoirs 118 and larger and smaller closure and dispensing component 109 are shown herein. Further, the skilled person is able to determine through trial and error alternative configurations and combinations of compressible sealing components 111, sample reservoirs 118, and closure and dispensing components 109 to achieve a desired volume of fluid being dispensed into the analysis portion 151 with appropriate operation of the closure and dispensing component 109 to reach the reaction chamber 117 (or chambers) without fluid leakage from the ventilation port 113 (if present).

Regarding the quantity of liquid dispensed, in some applications a selected or “predetermined” volume of liquid is to be dispensed into the analysis portion 151 of the fluidic cartridge so as to substantially fill the reaction chambers 117. In respective embodiments, the volume of liquid to be dispensed into the cartridge body 150 is 50-100 μl, 100 μl-150 μl, 150-200 μl, 150-200 μl, 200-250 μl, 250-300 μl, 300-350 μl, 350-400 μl, 400-450 μl, or 400-450 μl. However, it will be apparent to those skilled in the art that the dispensing cap 110 can be configured to dispense any practical volume or range of volumes of the sample fluid from the sample reservoir 118 into the reaction chamber(s) 117, in dependence on the predetermined pressure range in the sample reservoir 118 over the duration of the dispensing operation. For example, in some embodiments about 1000-1200 μl of air within the sample reservoir 118 is pressurised to an initial pressure of about 6-8 kPa, the pressure reducing during dispense of the sample fluid to a final pressure of about 4 kPa, dispensing a total volume of about 200 μl of the sample fluid into the analysis portion 151 to fill the 100 μl reaction chamber (and associated fluidic channels).

In some embodiments, the user can control how much of the sample fluid is dispensed into the analysis portion of the 151 of the fluidic cartridge. For example, the user can observe the fluid passing into a reaction chamber 117 through a viewing window 112 and then further screw down the dispensing cap 110 until a desired quantity of fluid has been dispensed. However, different users have different visual acuities and dexterities, and there may also be a delay between each screwing action and the resulting dispensing of a corresponding quantity of fluid. Therefore, in some embodiments, including the embodiment of FIG. 1, the dispensing cap 110 is configured so that the desired amount of fluid is dispensed into the cartridge when the dispensing closure is completely screwed on. Therefore, the user can simply screw down the dispensing cap 110 until it cannot be screwed down any further, and the dispensing cap 110 is then said to be fully closed. In the described embodiment, this occurs when the locking feature 115 of the dispensing cap 110 snaps into the locking recess 116 of the fluidic cartridge, preventing further rotation in either direction.

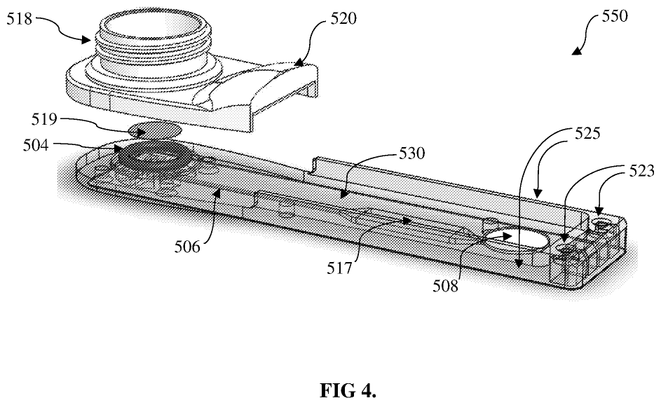

FIG. 4 is another example of a fluidic cartridge 550. Instead of four reaction chambers 117, as per the fluidic cartridge 150 of FIG. 1, the fluidic cartridge has only a single reaction chamber 517 and forms a continuous fluidic channel or tunnel 506 with the sample reservoir 518 when the opening in the wall or seal 519 of the sample reservoir 518 is broken. The cartridge body 530 further includes a ventilation port 508 to allow air to escape from the reaction chamber 517 as the fluid sample travels along the channel and into the reaction chamber 517. In some embodiments, the ventilation port comprises a ventilation membrane or filter 508 that is permeable to air but prevents fluid escape. The ventilation membrane or filter 508 may be, for example, a Porex PMV301 PTFE porous vent membrane. The ventilation membrane or filter 508 can be made of any suitable material that is air permeable but does not allow fluid to escape.

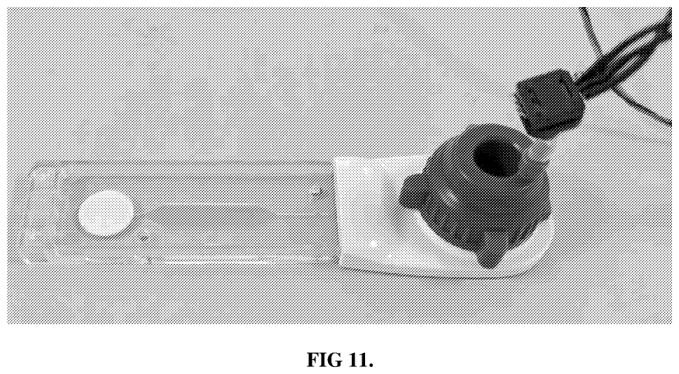

The pressure required to move the fluid from the sample reservoir 518 through to the reaction chamber 517 is determined to ensure that fluid passes through the channel but does not move out of the ventilation port 508 and/or overcome the ventilation membrane or filter. For example, FIG. 11 is a photographic image of a single channel fluidic cartridge and dispensing cap during pressure testing, showing how a dispensing cap can be provided with a pressure sensing port to enable pressure monitoring during operation so that the fluidic cartridge and dispensing cap can be configured to ensure precise pressure regulation for efficient and reliable fluid dispensing.

Recesses or detents 523 in the cartridge body 530 provide alignment points for the aligning the cartridge 550 within a diagnostic test reading instrument. For example, after insertion, the instrument lowers two balls into the detents 523 to hold the cartridge in position. Side rails 525 can also mechanically interface with the instrument to prevent the cartridge from lifting when inserted into the instrument.

The cartridge body 530 is transparent so that the reaction chamber 517 can be imaged from above without interfering with the instrument's spectral readings, for example, fluorescence from the reaction chamber 517. However, the cartridge body may alternatively be opaque and include a transparent viewing window, as per the cartridge 150 of FIG. 1.

An O-ring 504 forms a seal between the sample reservoir 518 and the cartridge body 530 to prevent leaking of sample fluid into the cartridge body. In an alternative embodiment, the fluidic cartridge body 530 and sample reservoir 518 are integrally formed, and an O-ring or other leak prevention feature is not required.

### Method for Using Dispensing Cap

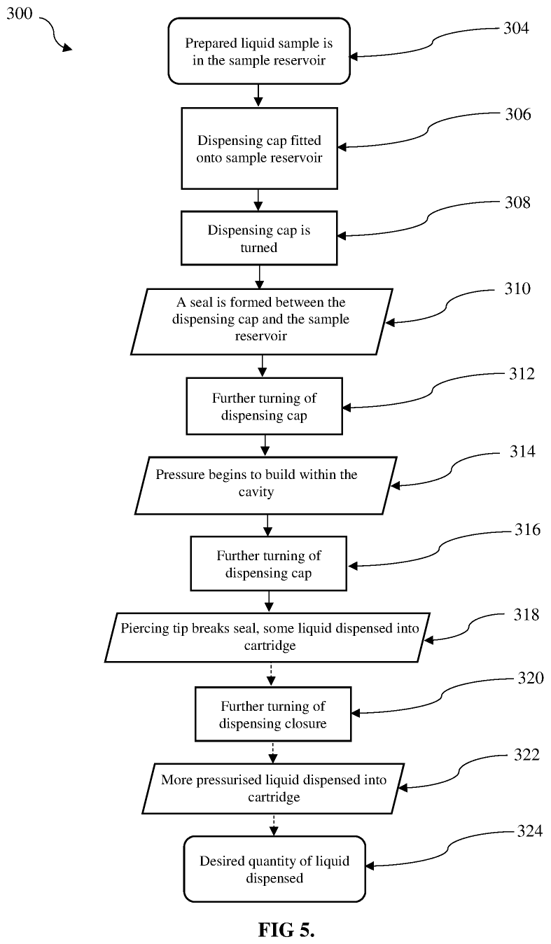

FIG. 5 is a flow diagram 300 of a method for dispensing fluid from a sample reservoir of a fluidic cartridge into an analysis portion of the fluidic cartridge, using a dispensing cap in accordance with an embodiment of the present invention. In the context of the embodiment described above, the process starts at step 304 after a sample fluid has been prepared, either within or outside of the fluidic cartridge, and is disposed in the sample reservoir 118. At step 306, the closure and dispensing component 109 of the dispensing cap 110 is then fitted onto the sample reservoir 118. At step 308, the dispensing cap 110 is partially screwed onto the fluidic cartridge by a person conducting the test.

At step 310, the compressible sealing component 111 comes into continuous contact with both the closure and dispensing component 109 and the sample reservoir 118, partially compressing the compressible sealing component 111 to form an airtight seal between the closure and dispensing component 109 and the sample reservoir 118, sealing the sample fluid within the sample reservoir 118 and creating an enclosed volume or cavity therein.

At step 314, of the dispensing cap 110 is further operated by the user by screwing it further onto the fluidic cartridge to further compress the compressible sealing component 111 and reduce the open volume within the sample reservoir and thereby pressurise the sealed sample reservoir 118. During step 316, the pressure continues to build with further operation of the dispensing cap 110 by the user. At step 318, the sample release component 202 makes an opening in the wall or seal 119 of the pressurised sample reservoir, and causes at least a portion of the sample fluid to be dispensed through the opening in the pressurised sample reservoir into the analysis portion 151 of the fluidic cartridge 150.

In some embodiments, further steps 320, 322 and 324 may be performed by the user. At step 320, further turning of the dispensing cap 110 at 320 continues to pressurise the sample reservoir 118, forcing more sample fluid to move into the analysis portion 151 of the fluidic cartridge 150. In some embodiments, the dispensing cap 110 can be freely rotated to dispense any available volume of the sample fluid, whereas in some other embodiments, detents or similar features are provided to allow the dispensing of multiple pre-determined volumes (which can be the same or different quantities) of the sample fluid. The process is complete when a desired quantity of sample fluid has been dispensed into the fluidic cartridge at step 324.

For clarity of description, steps 308 to 322 are depicted separately, however, the turning steps 308, 312, 316 and 320 also represent the continuous turning of the dispensing cap 110 by the person conducting the test, during which steps 310, 314, and 318 occur concurrently.

In the embodiment depicted by flow diagram 300, a seal is formed between the dispensing cap 110 and the sample reservoir 118 before the sample release component 202 creates an opening with the wall or seal 119 of the sample reservoir 118. However, in other embodiments the wall or seal 119 may be broken first. Typically, the sample reservoir 118 is connected to a reaction chamber 117 via an especially narrow fluidic channel (see FIG. 4), and therefore pressure will build inside the sample reservoir 118 and dispensing cap 110 as soon as the compressible scaling component 111 is engaged, regardless of whether an opening in the wall or seal 119 is made.

### Analysis Using Dispensing Cap and Fluidic Cartridge

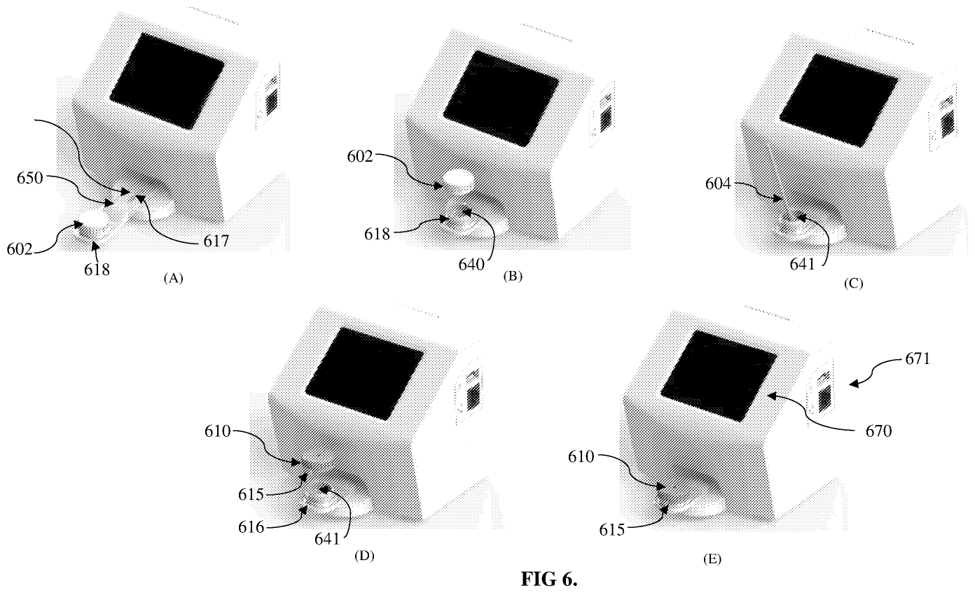

FIG. 6 illustrates an example workflow for performing a diagnostic test using a dispensing cap 610, fluidic cartridge 650, a diagnostic test reading instrument 660 and a dispensing cap 610 according to an embodiment of the present invention.

In FIG. 6A, the sample reservoir 618 of the cartridge 650 contains a pre-loaded sample preparation fluid. A shipping closure in the form of a shipping cap 602. and a wall or seal (not shown) at the base of the sample reservoir 618, together seal the sample preparation liquid 640 therein. In some embodiments, the dispensing cap is supplied as the shipping closure, and either not disposed in the scaling position, or includes a breakable tab component that prevents dispensing until the breakable tab component is removed. The fluidic cartridge 650 includes a viewing window 612 above a reaction chamber 617. Like the dispensing cap 610, the shipping cap 602 includes a scaling component (not shown) to prevent the sample preparation liquid from leaking. However, unlike the dispensing cap 110, the sealing component of the shipping cap 602 does not need to be, and typically is not, highly compressible like the compressible scaling component 111 of the dispensing cap 110 because the shipping cap 602 is not configured to pressurise the sealed sample reservoir 618. For example, in the described embodiments, the compressible scaling component 111 is a conventional and relatively incompressible o-ring. Also, the shipping cap 602 does not include an anti-rotation feature, because the shipping cap 602 needs to be removed prior to use in order to introduce the sample into the sample reservoir 618. As an alternative to a shipping cap 602, the sample reservoir 618 of other cartridges can be closed with, for example, a shipping closure in the form of a peelable seal or any other suitable removable closure (see below).

In FIG. 6B, the fluidic cartridge 650 has been inserted into the reader 660 so that the viewing window 612 above the reaction chamber 617 is aligned with the instrument's sensor (not shown). The shipping cap 602 is removed to expose the pre-loaded sample preparation liquid 640: in this case, an elution buffer solution. In FIG. 6C, a sample-containing swab 604 is stirred in the sample preparation liquid 640. The elution buffer releases the sample from the swab, resulting in an elution buffer and sample mixture 641, constituting a sample fluid. In alternative tests, a sample fluid is added to the sample preparation liquid 640 of the sample reservoir 618 using a pipette, syringe or other fluid transfer device, or a small material sample may be added to the preparation liquid 640 to form a sample fluid.

In FIG. 6D, the dispensing cap 610 is located above the sample reservoir 618 of the cartridge 650. The dispensing cap 610 includes a locking tab 615, which engages with a locking recess 616.

The sample fluid 641 is dispensed into the cartridge 650 by screwing the dispensing cap 610 onto the sample reservoir 618. As described above, as the dispensing cap 610 is screwed on, the dispensing mechanism of the dispensing cap 610 breaks the seal at the base of the sample reservoir 618, and the gasket of the dispensing cap 610 creates a seal between the sample reservoir 618 and the dispensing cap 610. Further rotation of the dispensing cap 610 compresses the gasket further and pressurises the air above the sample fluid 641, which forces the liquid 641 to move further into the cartridge 650: specifically, into the reaction chamber 617. The reaction chamber 617 is preloaded with soluble, dried or lyophilised reagents, which then react with the fluidic sample.

The dispensing cap 610 is configured to dispense the desired quantity of liquid into the reaction chamber 617 by continuing to screw the dispensing cap 610 onto the cartridge 650 until the locking tab 615 engages with the locking recess 616, as shown in FIG. 6E. The resulting locked dispensing cap 610 protects both a human operator and the instrument from contaminants and/or amplified genetic products during and after the test. The dispensing cap 610 may contain a detent feature to indicate that the user has screwed down the cap completely. The dispensing cap 610 can incorporate a one way latch such that the cap is locked in place to ensure that the dispensing cap 610 is not readily removed, and the cartridge remains sealed. Additionally, the tab or detent feature on the cap can be detected by the instrument to provide a signal to the instrument controller that the dispensing cap 610 is fully fitted.

As described above, the dispensing cap 610 can be designed or ‘tuned’ by determining the internal volume of air sealed within the dispensing cap 610 and sample reservoir 618 relative to the height of the compressible sealing component 111 of the dispensing cap 610. As will be appreciated, this internal air volume (or enclosed volume as described herein), changes with the quantity of sample fluid 641 within the sample reservoir 618. As is typically the case for diagnostic procedures, the quantity of pre-loaded sample preparation liquid 640 and the quantity of sample that will be added are known and reasonably consistent between tests. Cartridges are typically machine-filled with the pre-loaded sample preparation liquid 640, which ensures a consistent quantity of preparation liquid 640 between cartridges, and often the quantity of the sample to be added is small relative to the quantity of sample preparation liquid 640, and consequently slight variations will not materially affect dispensing.

Depending on the test, the instrument 660 can include a heated pad or surface or block underneath the base of the cartridge but in contact with the side walls of the sample reservoir 618 to bring the liquid in the reaction chamber 617 to a specific temperature for performing the test. The reader then reads and interprets the results of the test. In some embodiments, the instrument generates a movable magnetic field to cause one or more magnetically influenced metallic beads or balls within the sample reservoir 618 (or reaction chamber 617) to move correspondingly, and thereby provide additional mixing. The test results can be presented to a user via a display 670, and/or the results can be sent to an external device; for example, a printer or laptop using I/O ports 671. The display 670 can also be used to provide instructions to a user on how to perform the test.

Although each of the dispensing cap embodiments described herein screw onto a feature of a fluidic cartridge, the person skilled in the art will appreciate that other closure types may be used in other embodiments. For example, in some embodiments, the dispensing cap 610 is pressed to close rather than turned to close. The user presses the cap down until locking tabs on the cap or the sample reservoir lock the cap in position. In some embodiments, multiple such locking tabs, for example, a linear ratchet mechanism, allow for dispensing multiple volumes of one or more pre-determined volumes of sample fluid. Further, the dispensing cap 610 may be shaped to fit inside or otherwise engage with a feature of a cartridge, rather than fit over the sample reservoir 618 of a cartridge, as per the disclosed embodiments. In the described embodiments, the compressible sealing components 111 is a compressible gasket.

### Shipping Closures

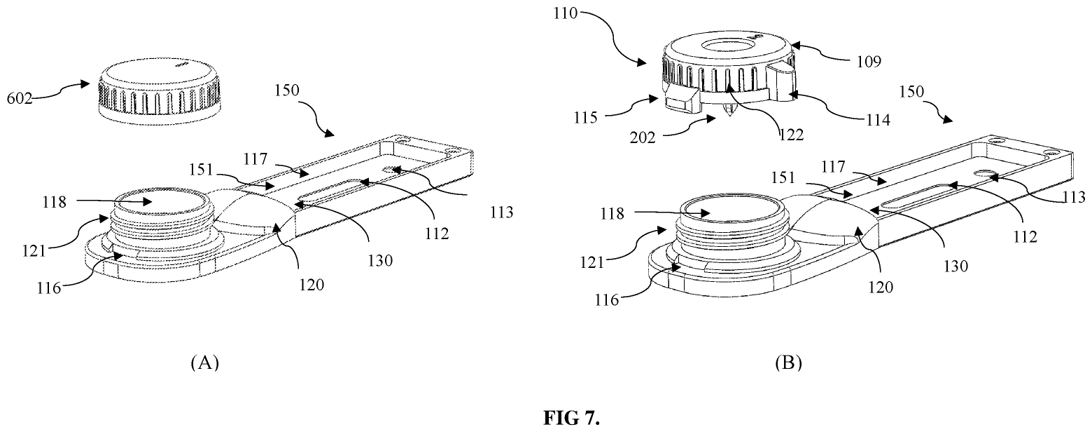

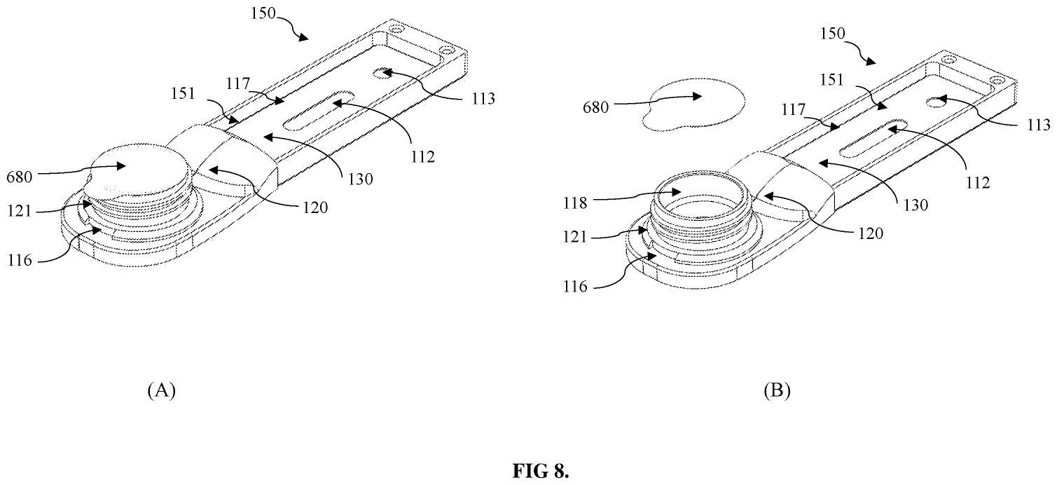

FIGS. 7 (A), and 8 (A) depict a single channel fluidic cartridge which is provided with a shipping closure respectively in the form of a cap 602, or a removable foil seal 680, and a dispensing cap 110 according to an embodiment of the present invention.

In the described embodiment, the shipping cap 602 of FIG. 7 (A) is formed from a bonded plastic laminate, but any suitable material known to the skilled person may be used in other embodiments. Similar to the dispensing cap 610 described above, the external surface of the shipping cap 602 of FIG. 7 (A) provides ridges to improve the user's grip. As described above, as an alternative to the shipping cap, a foil sheet may be attached to the top surface of the fluidic cartridge 150 as shown in FIG. 8 (A), thereby sealing the sample preparation liquid 640 within the sample reservoir 618. In either case, the shipping closure (e.g., either the shipping cap 602 or foil seal 680) is removed prior to use, and once the sample is added to the sample reservoir 618, it is replaced by the dispensing cap 610, as shown in FIG. 7 (B).

In FIG. 7A and FIG. 8A, the sample reservoir 118 of the cartridge 150 may contain a pre-loaded sample preparation fluid. The shipping closure (e.g., shipping cap 602 or removable seal 680) and a wall or seal (not shown) at the base of the sample reservoir 618 together seal these contents within the sample reservoir 618. As mentioned above, the fluidic cartridge 150 includes a viewing window 112 above a reaction chamber 130. Like the dispensing cap 610, the shipping cap 602 may include a sealing component (not shown) to prevent the sample preparation liquid from leaking. Alternatively, a removable seal, for example foil 680, without a compressible sealing component can be used. However, unlike the dispensing cap 110, the shipping closure (e.g., cap 602 or removable seal 680) is not configured to pressurise the sealed sample reservoir 618, and also does not include an anti-rotation feature, because the shipping closure (cap 602 or removable seal 680) needs to be removed prior to use in order to introduce the sample into the sample reservoir 618.

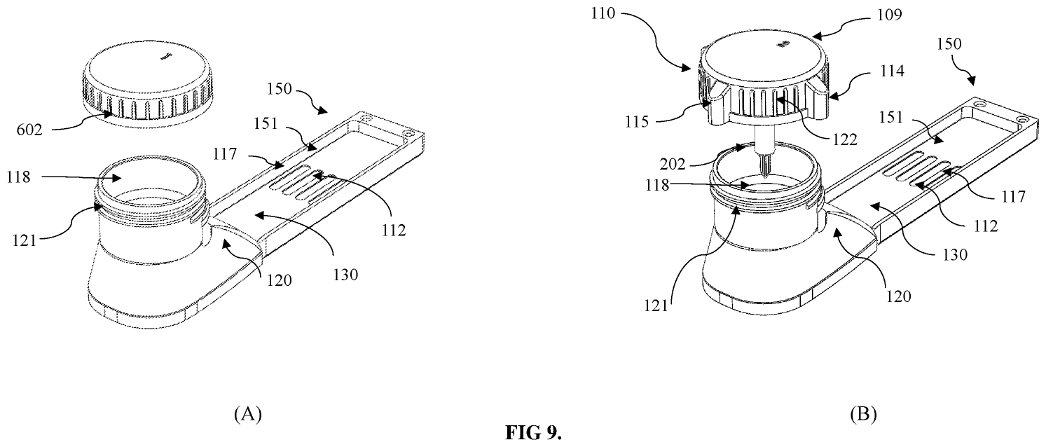

FIG. 9 depicts an example of a multi-channel fluidic cartridge which is provided with a shipping cap 602 and a dispensing cap 110 according to an embodiment of the present invention. Relative to the embodiments described above, the sample reservoir 118 is increased in height to provide for a higher volume of sample preparation liquid (not shown). The dispensing cap 110 can have a shorter height and a longer sample release component 202.

### Kits

The dispensing caps described herein are provided as a component of a fluidic cartridge kit, comprising:
a dispensing cap;
a fluidic cartridge having a sample reservoir and an analysis portion; and
a shipping closure to contain contents of the sample reservoir during transport.

In the case of a kit for use in detecting a nucleic acid in a sample, the kit can additionally comprise additional reagents, for example, lysis buffer or buffer for preparing the sample.

Optionally, a kit of the present disclosure is packaged with instructions for use in a method described herein.

As used herein “kit” means a collection of apparatus and reagents for performing an assay or test. A typical test kit for such tests includes a small box containing separate apparatus and reagents that a user individually opens and uses. Typically, such a test kit will contain a test tube or moulded consumable and separate powder and liquid reagents, along with a separate package that contains the test strip or cartridge.

The reagents may be supplied separately from the test cartridge, and are dispensed by the user into the cartridge following sampling. Only once the user has inserted the swab or sample into the liquid reagents do they then introduce the prepared sample to the cartridge. There is considerable error that may be introduced during this process by the user, and there is a time delay as the sample is washed in the reagents and introduced to the cartridge. The described kits address this problem by providing a single cartridge which is pre-filled with the solution for testing (e.g., lysis buffer). The user simply either removes the shipping cap or removable seal (e.g., foil covering), adds the sample (either by washing the swab or dispensing the blood sample into the input), and then attaches the dispensing closure to the input port, screwing down the dispensing closure until the locking tab 615 engages with a locking recess 616.

A kit may be “complete”, where all reagents needed for preparation and running of the test are provided. Alternatively, a kit may be “partial”, omitting certain reagents needed for operation. Both complete and partial kits may include additional reagents for sample preparation such as nucleic acid isolation.

### EXAMPLES

The present disclosure includes the following non-limiting examples.

### Dispensing Cap for Use in G6PD Testing

Drugs are available for treating latent malaria infection. However, these drugs are potentially harmful if administered to patients with low levels of glucose-6-phosphate dehydrogenase (G6PD) enzymatic activity. Accurate measurement of G6PD activity in blood also needs to be compensated for the level of haemoglobin (Hb). Therefore, a cost effective G6PD and Hb test is needed for the safe treatment of malaria, where the test is capable of determining the patient's G6PD level enzymatic activity and Hb level. This can be subsequently used to calculate, for example, the ratio of G6PD activity to Hb level. This ratio of G6PD enzyme activity to Hb level, also referred to as the “compensated G6PD value”, can then be used by clinicians to determine potential risks of drug applications or treatments.

G6PD is active in essentially all types of cells and is involved in protecting cells from oxidative stress. It is responsible for the first step in the pentose phosphate pathway, a series of chemical reactions that includes converting the oxidised form of nicotinamide adenine dinucleotide phosphate, referred to as NADP⁺, to NADPH. The rate at which this conversion occurs is known to be a measure of G6PD enzymatic activity in the sample. Methods for quantitative UV spectroscopy and UV spot testing are well described in the art. For example, global health organisation PATH has produced a commonly used guide to fluorescent spot testing for G6PD deficiency.

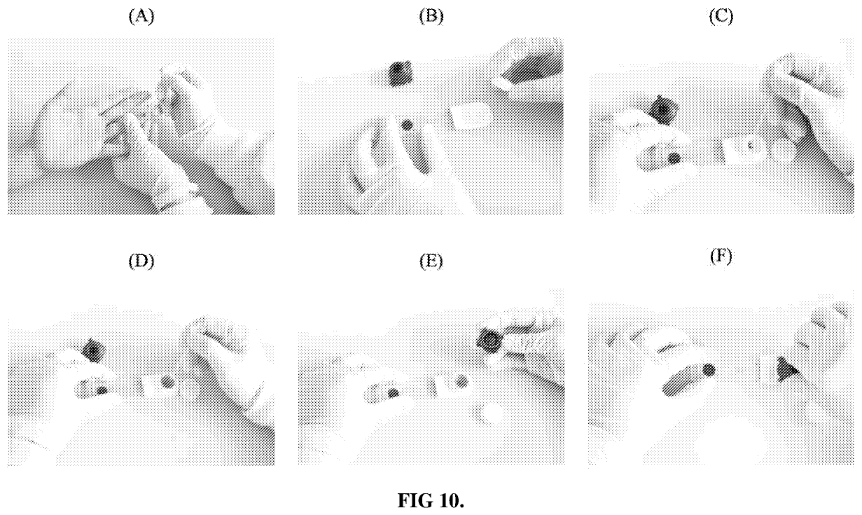

Embodiments of the dispensing cap of the present disclosure, for example, the dispensing cap 110 of FIG. 1 and FIG. 2, can be used to dispense a prepared blood sample into a fluidic cartridge, for example, the cartridge 550 of FIG. 5, for performing a G6PD and Hb test. FIG. 10 provides a series of images outlining the use of the dispensing cap in the G6PD and Hb test.

An example test procedure involves first collecting a blood sample from a patient's finger using a sterile lancet (FIG. 10A). The shipping cap is unscrewed and removed from the fluidic cartridge to expose the sample reservoir 518, which contains 500 μl of preloaded sample preparation buffer liquid (FIG. 10B). The sample preparation buffer, for example, is an aqueous solution with detergent, salt, hypertonic water, or other reagent configured to cause the lysis of red blood cells within the added sample, and to distribute the Hb throughout the solution. The reaction chamber 517 is preloaded with a soluble, dried or lyophilised reagent containing NADP⁺ and other reagents relevant to the assay. 5 μL of blood sample is transferred from the finger droplet to the sample reservoir 518 of the cartridge 550 using a simple transfer device, for example, a pipette (FIG. 10C and 10D). The person skilled in the art will appreciate the many different arrangements of volumes that may be used in alternative test configurations.

To start the test, the dispensing cap 110 is screwed on, the sample release component 202, preferably a piercing tip, creates an opening in the seal or wall 119, and approximately 100 μL of the blood sample diluted with the sample preparation buffer, is pumped from the sample reservoir 118 into the reaction chamber 517 (FIG. 10E and 10F).

Once inside the reaction chamber 517, the G6PD present in the sample starts to convert the preloaded NADP⁺ into NADPH. The NADPH generated by this reaction is a naturally fluorescent molecule whose fluorescence can be measured using known fluorescence-based readers. The measured level of fluorescence is proportional to the quantity of NADPH present in the reaction chamber 517 at the time the fluorescence response image is acquired. A set of NADPH levels is measured over time, and the slope/gradient of the NADPH level over time, i.e. the rate of change of the NADPH level, can be used to calculate the relative level of G6PD present in the sample. That is, the more G6PD present, the faster the preloaded NADP⁺ is converted to NADPH.

The above reaction is temperature dependent, and therefore the temperature of the cartridge 550, and more specifically the temperature of the reaction chamber 517, may be maintained at a specific known temperature for the duration of the test. For example, the temperature for the reaction may be selected to be between a minimum of 30° C. and maximum of 45° C. and maintained by the reading instrument. Alternatively, the temperature of the cartridge 550 is not controlled, but rather simply measured throughout the reaction and the NADPH rate calculations adjusted accordingly.

Where the instrument is capable of reading both colorimetric and fluorescent tests, the same reader can be used to obtain a Hb measurement. Hb, being red, absorbs blue or green light well, and consequently blue or green illumination with appropriate filtering is used to determine the Hb present in the sample. Alternatively, the cartridge 550 can be inserted into a second reader to obtain a Hb measurement.

Once both the G6PD and Hb levels in the sample have been determined, the compensated G6PD can be calculated by the instrument and communicated to the user.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Many modifications will be apparent to those skilled in the art without departing from the scope of the present invention.

1. A fluidic cartridge kit, including:
a fluidic cartridge having an analysis portion and a sample reservoir sealed with a removable shipping closure to contain contents of the sample reservoir during transport; and
a dispensing cap;
wherein the dispensing cap includes:
a closure and dispensing component configured to engage the dispensing cap to the fluidic cartridge and close an opening of the sample reservoir of the fluidic cartridge following removal of the shipping closure from the sample reservoir;
a compressible sealing component configured to form a seal between the closure and dispensing component and the sample reservoir and thereby seal the sample reservoir; and
a sample release component coupled to the closure and dispensing component and configured to make an opening in a wall of the sealed sample reservoir; and
wherein the closure and dispensing component is operable to engage the fluidic cartridge, close the sample reservoir, and compress the compressible sealing component therebetween to seal the sample reservoir, wherein the sample reservoir is partially filled with a sample fluid;
wherein the closure and dispensing component is further operable to compress the compressible sealing component and thereby pressurise the sealed sample reservoir and to cause the sample release component to make the opening in the wall of the pressurised sample reservoir; and
wherein the closure and dispensing component, the compressible sealing component and the sample release component are configured such that the pressurised sample reservoir is pressurised to a predetermined pressure range that causes at least a portion of the sample fluid to be dispensed through the opening in the pressurised sample reservoir into the analysis portion of the fluidic cartridge.

2. The fluidic cartridge kit of claim 1, further including a locking feature configured to lock the closure and dispensing component to prevent inadvertent release of sample fluid from the fluidic cartridge following dispensing.

3. The fluidic cartridge kit of claim 1, wherein the closure and dispensing component includes a screw thread configured to engage with a corresponding screw thread on an outer surface of the sample reservoir.

4. The fluidic cartridge kit of claim 1, wherein the compressible sealing component is a compressible gasket.

5. The fluidic cartridge kit of claim 4, wherein the compressible gasket comprises silicon foam.

6. The fluidic cartridge kit of claim 4, wherein the compressible gasket is composed of silicon foam.

7. The fluidic cartridge kit of claim 1, wherein a portion of the sample release component is configured to puncture and pass through the wall of the sample reservoir to make the opening therein.

8. The fluidic cartridge kit of claim 7, wherein a cross-sectional shape of the sample release component provides an opening to facilitate fluid flow through the opening in the wall of the sample reservoir with the sample release component through the opening.

9. The fluidic cartridge kit of claim 8, wherein the cross-sectional shape of the sample release component is a cross-shape.

10. The fluidic cartridge kit of claim 1, wherein the closure and dispensing component is variably operable to dispense a variable amount of the sample fluid into the analysis portion of the fluidic cartridge.

11. The fluidic cartridge kit of claim 1, wherein the closure and dispensing component is configured to dispense one or more predetermined amounts of the sample fluid into the analysis portion of the fluidic cartridge.

12. The fluidic cartridge kit of claim 11, wherein the closure and dispensing component has a morphology configured to provide a predetermined enclosed volume within the sample reservoir upon sealing, and to effect a predetermined reduction of that volume upon the further operation to pressurise the sample reservoir to a pressure that causes the predetermined amount of the sample fluid to be dispensed into the analysis portion of the fluidic cartridge.

13. The fluidic cartridge kit of claim 12, wherein the closure and dispensing component is domed to provide increased enclosed volume upon sealing of the sample reservoir.

14. The fluidic cartridge kit of claim 1, wherein the dispensing cap and sample reservoir are configured to provide a predetermined enclosed volume within the sample reservoir upon sealing, and to effect a predetermined reduction of that volume upon operation to pressurise the sample reservoir and cause the predetermined amount of the sample fluid to be dispensed into the analysis portion of the fluidic cartridge.

15. The fluidic cartridge kit of claim 13, wherein the sample reservoir is formed as a body of a plastic material with a second opening covered by a foil or seal composed of a different material, and wherein the sample release component makes the opening in the wall of the pressurised sample reservoir by puncturing the foil or seal.

16. The fluidic cartridge kit of claim 13, wherein the analysis portion of the fluidic cartridge comprises a reaction chamber configured to fill with the sample fluid upon the opening in the wall of the pressurised sample reservoir.

17. The fluidic cartridge kit of claim 13, wherein the analysis portion of the fluidic cartridge comprises multiple reaction chambers configured to fill simultaneously with the sample fluid upon the opening in the wall of the pressurised sample reservoir.

18. The fluidic cartridge kit of claim 13, wherein the analysis portion of the fluidic cartridge comprises multiple reaction chambers configured to fill sequentially with the sample fluid upon the opening in the wall of the pressurised sample reservoir.

19. The fluidic cartridge kit of claim 13, wherein the analysis portion of the fluidic cartridge comprises a ventilation port to allow air to escape from the reaction chamber(s).

20. The fluidic cartridge kit of claim 19, wherein the ventilation port comprises a ventilation membrane or filter that is permeable to air but prevents fluid escape.

21. The fluidic cartridge kit of claim 13, wherein the analysis portion comprises a non-transparent material covering the reaction chambers.

22. The fluidic cartridge kit of claim 13, wherein the analysis portion comprises a transparent material covering the reaction chambers.

23. The fluidic cartridge kit of claim 13, wherein the sample reservoir is integrally formed as a body of a plastic material, and the sample release component makes the opening in the wall of the pressurised sample reservoir by puncturing the plastic wall.

24. The fluidic cartridge kit of claim 1, wherein the shipping closure is a cap is made of a bonded plastic laminate.

25. The fluidic cartridge kit of claim 1, wherein the shipping closure is the dispensing cap.

26. The fluidic cartridge kit of claim 1, wherein the shipping closure is a foil sheet that seals the sample reservoir during transport.

27. The fluidic cartridge kit of claim 1, wherein the contents of the sample reservoir includes a lysis buffer.

28. A diagnostic test method, the method comprising:
introducing or forming a sample fluid containing a biological or environmental sample into or in the sample reservoir of the fluidic cartridge of claim 1;
applying the dispensing cap of claim 1 to the fluidic cartridge;
operating the closure and dispensing component to engage the closure and dispensing component to the fluidic cartridge, close the sample reservoir, and compress the compressible sealing component therebetween to seal the open volume and sample fluid within the sample reservoir; and
further operating the closure and dispensing component to further compress the compressible sealing component to reduce the enclosed volume within the sample reservoir and thereby pressurise the sealed sample reservoir and to cause the sample release component to make the opening in the wall of the pressurised sample reservoir, wherein the pressurised sample reservoir is pressurised to a predetermined pressure range that causes at least a portion of the sample fluid to be dispensed through the opening in the pressurised sample reservoir into the analysis portion of the fluidic cartridge.

29. The diagnostic test method of claim 28, wherein a single action by a user causes the portion of the sample fluid to be dispensed through the opening in the pressured sample reservoir into the analysis portion of the fluidic cartridge.
